(19) United States
(12) Patent Application Publication
(10) Pub. No.: US 2014/0308628 A1
(43) Pub. Date: Oct. 16, 2014

(54) METAL MATERIALS HAVING A SURFACE LAYER OF CALCIUM PHOSPHATE, AND METHODS FOR PREPARING SAME

(71) Applicant: CENTRE NATIONAL DE LA RECHERCHE SCIENTIFIQUE (C.N.R.S), Paris (FR)

(72) Inventors: Adele Carradò, Strasbourg (FR); Geneviève Pourroy, Schiltigheim (FR); Wafa Abdel-Fattah, Le Caire (EG)

(73) Assignee: Centre National de la Recherche Scientifique (C.N.R.S.), Paris (FR)

(21) Appl. No.: 14/357,347

(22) PCT Filed: Nov. 12, 2012
(86) PCT No.: PCT/EP2012/072407

§ 371 Date: May 9, 2014

(30) Foreign Application Priority Data
Nov. 10, 2011 (FR) 11 60288

(51) Int. Cl. A61C 8/00; A61F 2/30

(52) U.S. Cl. CPC A61C 8/0013; A61F 2/30771; A61F 2002/3093
USPC 433/173; 623/23.51; 623/18.11; 433/201.1; 428/560; 427/2.27

(57) ABSTRACT

The present invention relates to a multi-layer material comprising a metal or metal alloy substrate, the metal or alloy substrate being coated with an intermediate layer comprising at least one ceramic or crystalline, or partially crystalline, structure, including a metal or a metal alloy, said intermediate layer being coated with a layer of calcium phosphate having a cellular nanometric structure, and uses thereof.

The present invention relates to the method for preparing such a material by autocatalytic deposition of a layer of calcium phosphate comprising a cellular nanometric surface structure.

### FIELD OF THE INVENTION

The present invention relates to a multi-layer material comprising a metal or metal alloy substrate, said metal or alloy substrate being coated with an intermediate layer comprising at least one ceramic or crystalline, or partially crystalline, structure, including a metal or a metal alloy, said intermediate layer being coated with a layer of calcium phosphate having a cellular nanometric structure, and uses thereof.

The invention also relates to methods for preparing such a material, said method comprising the autocatalytic deposition of the layer of calcium phosphate, which is optionally followed by a growth phase of the calcium phosphate layer.

In particular, the invention relates to the field of medical implants (or medical prostheses), and in particular bone implants.

### BACKGROUND OF THE INVENTION

Medical implants are generally made from a metal or alloy compatible with the human body. However, this compatibility requires improvement, particularly in terms of its compatibility with bone, and in particular to improve osteoblast growth at least at the implant/bone interface.

Considerable work has been done on the formation of a bioactive deposit on metal or nonmetal substrates intended to be implanted in humans with the aim of combining the mechanical properties of the substrate and the bioactivity of the layer. Titanium and its alloys are excellent metal materials for dental and orthopedic surgery applications, due to the high mechanical strength, the low elasticity modulus, their high resistance to corrosion and excellent biocompatibility. Hydroxyapatite (HA) is the ceramic generally used as bioactive layer, since it can bond chemically with the bone. It thus makes implants with a base of titanium or its alloys more compatible and improves osteoblast growth.

To produce hydroxyapatite, the metal substances used to make the implants are submerged in a bath. Several baths been tested, but the results do not always agree. Among these treatments, the alkaline treatment is the most common and appears to be the most effective. More recently, Takeuchi et al. (Acid pretreatment of titanium implants, Biomaterials 24 (2003) 1821-1827) and Jonasova et al. (Biomimetic apatite formation on chemically treated titanium, Biomaterials 25 (2004) 1187-1194) indicate that the combination of acid and alkaline treatments could be more effective to form a layer similar to bone apatite on the surface of the titanium when the substrate is submerged in a solution of simulated body fluid (SBF).

Currently, bone prostheses are made by plasma torch to obtain a thick hydroxyapatite layer. However, these prostheses suffer from the problem of stripping of the hydroxyapatite layer.

Furthermore, it is also known that an autocatalytic deposition may be done in the case of biomaterials. However, this technique has only been used on polymer-based biomaterials, but not in the case of metallic or metal alloys (Leonor and Reis, An innovative autocatalytic deposition route to produce calcium-phosphate coatings on polymeric biomaterials, J. Material Science: Materials in Medicine, 2003, 14, 135). There is therefore no teaching on the possibility of performing such autocatalytic depositions on metals or alloys, in particular for medical use.

### SUMMARY OF THE INVENTION

At this time, the techniques used to improve compatibility between metals or metal alloys for human implants and bone must be improved.

Thus, the present invention aims to provide a new material improving the compatibility of the metals or alloys with the bone, in particular when it involves titanium or a titanium alloy.

The present invention aims to provide a porous coating that may be impregnated by medications (antibacterial agents, growth factor, etc.).

The present invention aims to improve the preparation of implant materials requiring good compatibility with the bone.

The present invention also aims to improve the mechanical properties of materials usable in the medical field such as implants or prostheses, and to improve the bioactivity of their surfaces. The present invention also aims to improve the lifespan of such materials.

The present invention also aims to provide an inexpensive, reliable solution that is usable on an industrial scale.

Thus, the present invention relates to a multi-layer material comprising a metal substrate or a metal alloy, the metal substrate or alloys being coated with an intermediate layer comprising at least one ceramic or one crystalline, or partially crystalline, structure, including a metal or a metal alloy such as, for example, an oxide or nitride of a metal or an alloy, said intermediate layer being coated with a layer of calcium phosphate comprising a cellular nanometric structure on the surface.

### BRIEF DESCRIPTION OF THE DRAWINGS

FIG. 1 diagrammatically shows two alternatives of the inventive method.

FIG. 2 diagrammatically shows the layers of the material of the invention.

FIGS. 3(a and b) show photographs by FESEM (Field Emission Scanning Electron Microscopy) after chemical etching of the substrate.

FIG. 4 shows a diagrammatic view of a device for alkaline chemical and heat treatment according to one alternative of the invention.

FIG. 5(a-c) show photographs of an intermediate layer by FESEM after alkaline chemical and heat treatment according to one alternative of the invention.

FIG. 6 shows a diagrammatic view of a device for deposition by autocatalytic bath according to one alternative of the invention.

FIG. 7 shows FESEM photographs of calcium phosphate layers obtained by different autocatalytic baths after chemical treatment.

FIG. 8(a-c) show FESEM photographs of calcium phosphate layers obtained by different autocatalytic baths deposited on a layer of titanium nitride deposited by PLD.

FIG. 9(a-c) show FESEM photographs of calcium phosphate layers obtained by different autocatalytic baths deposited on a layer of titanium dioxide deposited by PLD.

FIG. 10 shows a FESEM photograph (top) of the calcium phosphate layer obtained by spin coating and (bottom) the graph obtained by EDS-X analysis (energy-dispersive analysis).

FIG. 11 shows a FESEM photograph (top) of the calcium phosphate layer obtained by dip coating and (bottom) the graph obtained by EDS-X analysis.

FIGS. 12 and 13 show the cell viability on substrates made from Ti6Al4V (commercial) treated by autocatalytic baths (3 hours) with PdCl₂ (FIG. 12) as catalyst and by autocatalytic baths (2 hours) with AgCl (FIG. 13) as catalyst.

### DETAILED DESCRIPTION OF THE PREFERRED EMBODIMENT

“Cellular nanometric structure” refers to a structure having visible surface pores (observed using Scanning Electron Microscopy) with an average diameter smaller than 1 μm. These pores coarsely form cells similar to the natural structure of a cancellous bone. These cells comprise relatively thin and flat walls. When reference is made to the structure of a bone, we are in particular referring to that of a cancellous bone. As emerges from the examples and figures, the material according to the present invention with a nanometric cellular surface structure is obtained without growth treatment of the layer of calcium phosphate in the presence of a simulated body fluid (SBF), or before such a growth treatment. The material according to the present invention is therefore very advantageous, since it has a cellular nanometric structure comparable to the natural structure of a cancellous bone (“bone-like”) without additional growth treatment of the layer of calcium phosphate in the presence of an SBF.

The metal or alloy substrate may in turn be a layer on another substrate.

It is preferable in the invention to use, as metal or alloy for the intermediate layer, one or more metals identical to at least one of the metals used for the substrate.

Among the metals according to the invention, it is preferable to use a metal chosen from among titanium or an alloy comprising titanium. Such materials are typically medical alloys, and in particular the following alloys: Ti6Al4V, NiTi (Nitinol®), X2CrNiMo18-15-3, X4CrNiMnMoN21-9-4, titanium-zirconium Ti-6Al-7Nb, Ti-5Al-2.5Fe, Ti-13Nb-13Zr, and Ti-15Mo-3Nb, stainless steel, for example of type 316, 316L, or 304, and in particular of type X2CrNiMo18-14-3, X2CrNiMo17-12-2, X5CrNiMo17-12-2, or X5CrNi18-10.

Preferably, a substrate is used comprising or made up of titanium or an alloy comprising titanium, like those cited above, and an intermediate layer comprising titanium.

The metal or alloy substrate advantageously has a roughness of less than 800 nm, and preferably less than 500 nm.

For the intermediate layer, it is preferable to use a ceramic or crystalline, or partially crystalline, structure comprising titanium.

“Ceramic or crystalline, or partially crystalline, structure including a metal or a metal alloy” in particular refers to the oxides, nitrides of the metal(s) or alloy(s) according to the invention.

The intermediate layer comprises or is preferably made up of sodium titanate (Na₂Ti₅O₁₁), titanium dioxide, titanium nitride, or a combination thereof. It is preferable for the intermediate layer to comprise or be made up of titanium nitride, and still more preferably for it to comprise or be made up of sodium titanate (Na₂Ti₅O₁₁).

According to one preferred alternative, the intermediate layer comprises a cellular nanometric structure. Preferably, the structure comprises average pore diameters smaller than 100 nm, observed by scanning electron microscopy.

According to one alternative, the intermediate layer has a thickness of 50 nm to 10 μm. This intermediate layer optionally has substantially spherical agglomerates with a diameter of from 1 to 3 micrometers, but the nanometric layer remains visible by regions.

According to one alternative, the intermediate layer has a thickness of 100 to 500 nm. According to this alternative, substantially spherical agglomerates are missing or substantially missing. The intermediate layer has a smooth surface that hugs the morphology of the metal substrate or metal alloy substrate.

Furthermore, the layer of calcium phosphate advantageously has a porosity of 50 to 400 nm (average pore diameters observed by Scanning Electron Microscope). The layer of calcium phosphate typically has a thickness of 100 nm to 100 μm, and preferably from 1 to 50 μm.

The layer of calcium phosphate is advantageously obtained by autocatalytic deposition, which is optionally followed by a growth phase of the calcium phosphate.

According to another aspect, the invention relates to a method for preparing a multilayer material comprising a metal or metal alloy substrate, and an intermediate layer comprising a ceramic or crystalline, or partially crystalline, structure including a metal or a metal alloy, for example such as an oxide or nitride of a metal or alloy, in which said method comprises:

(i) the mechanical polishing of a metal or metal alloy substrate,

(ii) chemical etching to remove any native surface oxides of the substrate;

(iii) producing an intermediate layer comprising at least one ceramic or crystalline, or partially crystalline, structure, including a metal or metal alloy on the surface of the substrate; and

(iv) the deposition, on the intermediate layer of the material obtained in step (iii), of a layer of calcium phosphate comprising a cellular nanometric surface structure.

According to a first alternative, the method comprises, in steps (ii) and (iii):

(a1) chemical etching to remove the native surface oxides by putting the polished surface in contact with an aqueous solution of hydrochloric acid and nitric acid;

(b1) placing the material in contact, for example by submersion, with an alkaline solution to generate a deposit, on the surface of the substrate, of an intermediate layer of a ceramic or crystalline, or partially crystalline, structure, including a metal or a metal alloy, and preferably titanium, then preferably washing and drying the material; and

(c1) heat treatment of the material.

According to a second alternative, the method comprises, in steps (ii) and (iii):

(a2) chemical etching to remove the native surface oxides, refine the porosity and passivate the surface of the substrate, to prepare the surface of the substrate for step (b2); and

(b2) producing a layer of a ceramic or crystalline, or partially crystalline, structure, including a metal or a metal alloy, and preferably titanium, by pulsed laser deposition (PLD) on the surface of the substrate.

Polishing—Step (i)

The mechanical polishing in step (i) is preferably done by using one or more abrasive compounds, for example silicon carbide, so that the substrate has an arithmetic roughness (Ra) of less than 0.5 μm, and preferably less than or equal to 0.2 μm.

The mechanical polishing treatment according to the invention, unlike what is generally taught by the prior art, makes it possible to decrease the roughness of the surface state of the metal or the alloy used. It has been discovered that if the roughness is too high (for example, Ra=2 μm), certain parts of the metal or the alloy could still be visible after the calcium phosphate deposition. The inventors have overcome this drawback of the prior art. In particular, an excessive roughness of the metal substrate will decrease the adhesion capacity of the cells on the implant. However, the invention aims to present a more natural implant surface to improve the adhesion and growth of osteoblasts.

Chemical Etching—Step (ii)

Before preparation of the intermediate layer (step (iii)) making it possible to improve the cohesion between the intermediate layer and the substrate, a surface treatment is applied to the substrate to improve the surface state of the metal or alloy of the substrate. This treatment in particular makes it possible to at least partially eliminate the native surface oxides. According to the alternative of the invention to prepare the intermediate layer in step (iii), the surface treatment of the substrate may be different. Thus, it is preferable to perform the following treatment to prepare the intermediate layer by chemical treatment:

Etching—Step (ii)/(a1)

Generally, the chemical etching step (a1) comprises the use of a combination of nitric acid and hydrochloric acid for a length of time preferably shorter than 8 minutes and preferably shorter than 5 minutes, and still more preferably for 2 to 3 minutes. Preferably, Kroll's reagent will be used.

Etching—Step (ii)/(a2)

To prepare the surface state of the substrate for pulsed laser deposition (PLD), the following treatment will preferably be done:

The chemical etching step (a2) is advantageously done by placing the material in contact with an alkaline solution comprising an oxidizing agent, and preferably with a solution of sodium hydroxide and hydrogen peroxide, this step preferably being done at a temperature comprised between 60 and 100° C., preferably for at least 5 minutes.

Step (a2) advantageously comprises placing the product in contact with an oxalic acid solution, preferably at a temperature comprised between 70 and 100° C., preferably for at least 10 minutes, to produce a microporous surface.

Step (a2) preferably comprises passivation of the surface of the substrate using nitric acid.

Preferably, all three of the treatments above (etching, oxalic acid and passivation) will be done to prepare the substrate for the PLD.

At the end of step (ii) (a1 or a2), one or more washing operations with water will preferably be done, then the material is dried.

Production of the Intermediate Layer—Step (iii)

As indicated above, two alternatives are preferred in the invention, namely a chemical preparation (purely chemical) and a preparation including pulsed laser deposition (PLD).

This step in particular aims to improve the cohesion between the substrate and the layer of calcium phosphate. This intermediate layer is advantageous to prepare a cellular nanometric calcium phosphate structure with a satisfactory thickness, which does not have the stripping drawback of the prior art. The layer of TiN deposited on the titanium by PLD is characterized by a nanometric crystallite size and columnar growth thereof. It may increase the hardness of the prepared intermediate layer. The films have been adhered to the substrates simply using the adhesive strip test (epoxide type). No plucking (unsticking) or cracking was observed for the deposited films. The lack of stripping of the layer of calcium phosphate was observed by scanning electron microscopy.

Chemical Preparation of the Intermediate Layer (b1 and c1)

This step preferably comprises treatment with an alkaline solution, preferably sodium hydroxide, at a concentration preferably of 5M to 15M, and preferably approximately 10M. This treatment is preferably done at a temperature comprised between 40 and 80° C., preferably at a temperature of approximately 60° C. The material is typically placed in contact with the alkaline solution for 1 hour to 2 days, and contact for 18 to 30 hours, and advantageously 24 hours, is preferable.

According to one alternative, this layer comprises sodium and titanate ions, forming a layer of sodium titanate. The heat treatment step (c1) is preferably done at a temperature comprised between 620° C. and 650° C., preferably between 625° C. and 635° C. for a sufficient length of time to dehydrate and crystallize the layer obtained in fine in step (iii). The treatment according to step (b1), followed by a heat treatment according to step (c1), leads to the formation of a partially crystalline porous layer, for example of sodium titanate, on the surface of the sample.

The layer obtained has a heterogeneous structure made up of spherical agglomerates with a diameter of 1 to 2 microns deposited on a cellular nanometric porous structure very similar to the structure of a bone with pore diameters smaller than 100 nm on average.

Preparation of the Intermediate Layer by PLD (b2)

For this alternative of the invention, step (b2) comprises the production of a layer of 100 to 500 nm of metal nitride or dioxide, preferably titanium nitride or dioxide, by pulsed laser deposition (PLD) on the surface of the substrate.

It is preferable to heat the material during PLD. The temperature may be kept above 580° C., for example at 600° C.

PLD is preferred to chemical deposition because the metal or metal alloy surface is much more homogenous than by chemical deposition, and has a lower surface roughness, consequently favoring the deposition and growth of calcium phosphate accordingly (steps (iv) and (v)). The spheroids observed by chemical deposition are missing or substantially missing by PLD. However, the cost of PLD treatment is higher.

Furthermore, according to one alternative, PLD makes it possible to deposit an intermediate layer of titanium dioxide or titanium nitride, which has advantageous mechanical properties. In particular, titanium nitride makes it possible to improve the mechanical properties of the layer of calcium phosphate by improving its adhesion to the layer of titanium nitride. Furthermore, the layer of titanium nitride has a strong fatigue strength, a hardness, a Young's modulus, and a rigidity that are very high, as well as a low mechanical wearing coefficient, close to those specific to human bone. The layer of titanium dioxide has very good bioactive properties and makes it possible to prevent bacterial infection.

Kokubo et al. (Formation of biologically active bone-like apatite on metals and polymers by a biomimetic process, Thermochimica Acta, 280/281 (1996) 479-490) describes a biomimetic method for apatite growth on metal or polymers. The deposition obtained is easily metabolized by the cells of the bone. This deposition leads to a spheroid surface having a diameter of several microns, and typically 5 to 10 microns, different from the natural surface of a bone. However, the invention aims to provide a material whereof the structure is close to the natural structure of a bone.

It has been discovered by this invention that by performing a prior deposition of calcium phosphate on an intermediate layer of a metal or alloy by autocatalytic bath, it is possible to improve the structure of the calcium phosphate layer to mimic the natural structure of a bone, and therefore to improve the structure of metal implants for integration into the bone.

Calcium Phosphate Deposition—Steps (iv)

Advantageously, step (iv) is done by placing the material, preferably by submersion, in a solution comprising calcium and phosphate ions for autocatalytic deposition in the intermediate layer, in contact with a calcium phosphate layer comprising a cellular nanometric structure on the surface; or this is done by depositing a calcium phosphate sol gel on the intermediate layer to obtain a calcium phosphate layer comprising a cellular nanometric structure on the surface.

(a) Deposition by Autocatalytic Bath—Steps (iv)

According to one particular embodiment, the autocatalytic bath comprises an oxidizing bath, an acid bath or an alkaline bath.

Advantageously, step (iv) is carried out at a temperature comprised between 50° C. and 100° C., and preferably between 60° C. and 80° C.

Step (iv) is preferably done: (a) at a temperature comprised between 50° C. and 70° C., and preferably approximately 60° C., in an alkaline bath, preferably at a pH comprised between 8 and 10, and preferably at a pH of about 9.2; or (b) at a temperature between 60° C. and 80° C., and preferably approximately 70° C., in an oxidizing bath, preferably at a pH of about 7; or (c) at a temperature between 70° C. and 90° C., and preferably about 80° C., in an acid bath, preferably at a pH comprised between 4 and 6, and preferably at a pH of about 5.3.

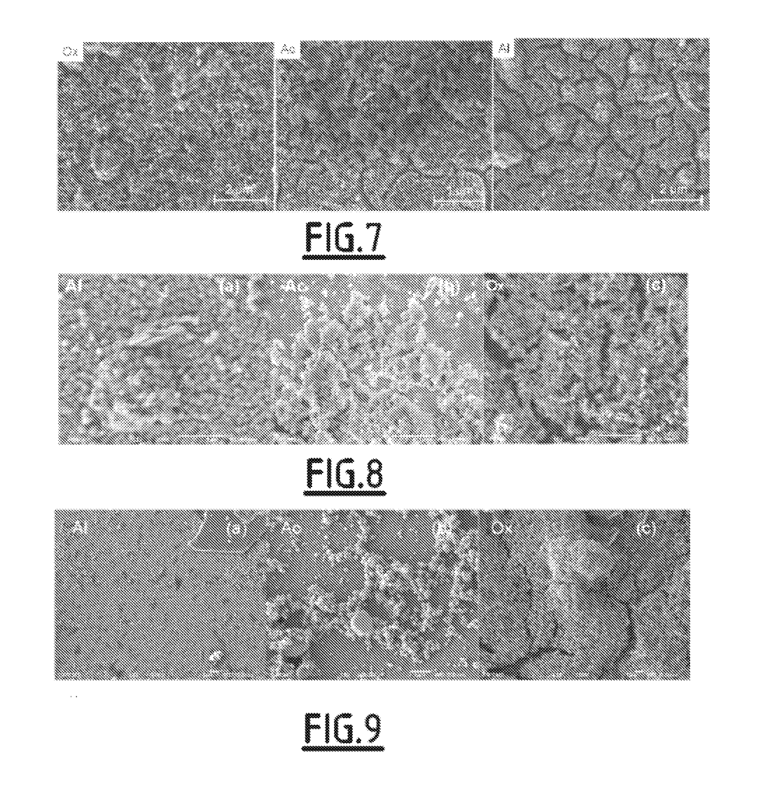

Depositing calcium phosphate by autocatalytic bath makes it possible to improve the growth of the calcium phosphate layer, and in particular to produce a layer having a structure very similar to that of the bone. It can, for example, be seen in FIGS. 7, 8 and 9.

Growth of a layer is observed whereof the structure is different depending on the autocatalytic bath used. The alkaline and oxidizing baths lead to similar structures with pores whereof the diameter (average diameter measured on images obtained by scanning electron microscope) is preferably comprised between 100 and 200 nm, similar to the porous structure of the bone. An oxidizing autocatalytic bath preferably contains calcium, pyrophosphate, and an oxidizing agent. An alkaline autocatalytic bath preferably contains pyrophosphate, hypophosphite and calcium.

An acid autocatalytic bath generally leads to spherical aggregates in the vicinity of several microns. An acid autocatalytic bath preferably contains calcium, hypophosphite and an organic acid. An organic acid is preferably chosen among the mono, di or tri-acids with a linear or branched hydrocarbon chain of 1 to 10 carbon atoms, optionally containing or substituted by one or more functions or substitutes.

The autocatalytic baths comprise palladium or a palladium compound as catalyst, or silver or a silver compound as catalyst, and for example palladium chloride or silver chloride.

According to one alternative of the invention, the oxidizing bath comprises calcium chloride, sodium pyrophosphate, hydrogen peroxide, and palladium chloride or silver chloride. According to one alternative, the acid bath comprises calcium chloride, sodium fluoride, succinic acid, sodium hypophosphite, and palladium chloride or silver chloride.

According to one alternative, the alkaline bath comprises sodium chloride, sodium pyrophosphate, sodium hypophosphite, and palladium chloride or silver chloride.

Preferably, the calcium chloride concentration is comprised between 1 and 50 g/L. Preferably, the sodium pyrophosphate concentration is comprised between 1 and 100 g/L. Preferably, the hydrogen peroxide concentration is comprised between 0 and 50 g/L. Preferably, the sodium hypophosphite concentration is comprised between 10 and 50 g/L. Preferably, the organic acid concentration is comprised between 1 and 20 g/L.

(b) Deposition by Sol Gel Preparation—Step (iv)

According to one alternative, the layer of calcium phosphate may be prepared by depositing a gel obtained using a sol gel process or method.

Sol gel methods for preparing a calcium phosphate gel from a calcium phosphate solution are known in the prior art.

Usable methods in particular include the deposition of a gel by spin coating, or by dip coating on the substrate obtained after step (iii).

The deposition according to this alternative of the invention (sol gel) makes it possible to obtain a layer of calcium phosphate generally of 500 nm to 50 μm. More specifically, depositing a gel by spin coating generally makes it possible to obtain a calcium phosphate thickness comprised between 0.5 and 10 μm; depositing a gel by dip coating in general makes it possible to obtain a calcium phosphate thickness comprised between 0.5 and 20 μm. It is easier to control the thickness of the layer formed by spin coating, while the layer obtained by dip coating is thicker.

Growth of the Calcium Phosphate Layer—Steps (v)

Advantageously, the method comprises a step (v) for growth of the calcium phosphate layer by placing the material in contact with a simulated body fluid (SBF). According to one alternative, the simulated body fluid may reproduce (in vitro) human blood plasma (with ion concentrations approximately equal to those of human blood plasma) in order to measure the bioactivity of the layer of calcium phosphate on the substrate.

The simulated body fluid advantageously comprises ions: sodium, carbonate, phosphate, magnesium, chloride, calcium and sulfate.

The placement in contact is preferably done for at least 1 day, and preferably for 4 to 15 days.

The calcium phosphate layer preferably has a thickness from 100 nm to 100 μm, and still more preferably from 10 to 100 μm.

Advantageously, the calcium phosphate layer has a porosity of 50 to 100 nm, reduced relative to that of step (iv).

Phosphate and carbonate formation is observed (observation by infrared spectrometry). The calcium and phosphate concentration of the SBF solution increases in the first 2 days. After 7 to 14 days, the calcium and phosphorus concentration of the SBF solution decreases, showing absorption of those cations onto the substrate.

After treatment by autocatalytic bath (step (iv)), in the presence of SBF (step (v)), a growth of the calcium phosphate layer is observed that may go from several hundred nanometers to several tens of microns. The formation process for these deposits is very similar to that which leads to the natural formation of the bone. This is therefore a very significant advantage of the present invention. Significant thicknesses are obtained, in particular using an inexpensive method adapted to complex sample geometries (implants, prostheses or others). The growth is done by biomimetism of the bone growth. The morphology of the calcium phosphate layer is adapted to the cell growth and impregnation by active agents. To allow the osteoblasts to better adhere to the surface and grow, the layer of calcium phosphate may contain chemical elements improving cell adhesion and/or cell growth. Thus, according to one alternative, the layer of calcium phosphate comprises one or more compounds improving the adhesion and/or growth of the osteoblasts.

The layer of calcium phosphate obtained according to the present invention allows it to be impregnated by such compounds. These compounds are known by those skilled in the art. They are in particular active agents, such as one or more antibacterial agents (for example, silver ions Ag⁺ (W. Chen et al. In vitro antibacterial and biological properties of magnetron co-sputtered silver-containing hydroxyapatite coating, Biomaterials, 27, 32, 2006, pp 5512-5517), Furanone (J. K. Baveja et al. Furanones as potential antibacterial coatings on biomaterials, Biomaterials, 25, 20, September 2004, pp 5003-5012) versus Staphylococcus epidermis and Staphylococcus aureus and/or one or more growth hormones (transforming growth factor (TGF-β1), parathyroid hormone (PTH) and prostaglandin E2 (PGE2) (K. Anselme Osteoblast adhesion on biomaterials, Biomaterials, 21, 7, 2000, pp 667-681). The invention also makes it possible to incorporate active agents into the calcium phosphate layer, such as medications (antibiotics, etc.), for example to fight infections. These medications are known by those skilled in the art.

Furthermore, the invention makes it possible to avoid the problem of stripping of the calcium phosphate layer, while having a satisfactory thickness of the calcium phosphate layer. The material according to the invention has a lower crystallinity than a thick layer of hydroxyapatite formed by plasma torch, which is more favorable to the osteoblast adhesion, proliferation and exchanges with the surrounding medium. The layer is partially amorphous because (1) the deposits have been done at low temperatures, and (2) there has not been any recrystallization by heat treatments.

The layer of calcium phosphate according to the invention for example in particular comprises calcium carbonate (CaCO₃) associated with hydroxyapatite, monocalcium phosphate Ca(H₂PO₄)₂, or dicalcium phosphate (CaHPO₄).

The invention also relates to a multilayer material that may be obtained using the inventive method, according to any one of its alternatives and embodiments, including any combinations thereof.

The invention also relates to an implant or a prosthesis for a bone structure comprising a material as defined in the present description. In particular, the invention relates to a bone implant, or a dental implant.

The invention also relates to the use of a multilayer material, as defined in the present description, to prepare an implant or prosthesis for a bone or dental structure.

The invention also relates to an implant composition for a bone structure comprising or made up of a multilayer material as defined in the present description, and in particular to be used in the surgical treatment of a human being.

Advantageously, said composition is used to replace an articular bone end, for example for bone surgery in a hip, knee, shoulder, elbow, ankle, wrist, fingers and/or toe, or for dental surgery.

Other aims, features and advantages of the invention will appear clearly to one skilled in the art after reading the following explanatory description done in reference to examples provided solely as an illustration and that are not in any way limiting on the scope of the invention.

The examples are an integral part of this invention, and any feature appearing to be novel relative to any prior state of the art from the description in its entirety, including the examples, is an integral part of the invention in terms of its function and generality.

Thus, each example has a general scope.

Furthermore, in the examples, all of the percentages are given by weight unless otherwise indicated, the temperature is expressed in degrees Celsius unless otherwise indicated, and the pressure is the atmospheric pressure unless otherwise indicated.

### EXAMPLES

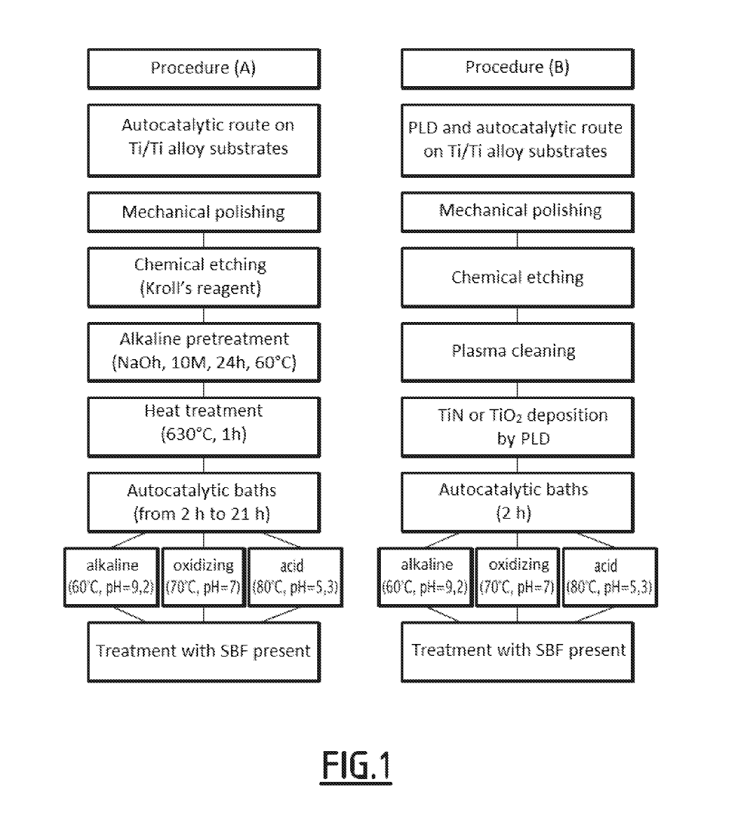

FIG. 1 diagrammatically shows a block diagram of two alternatives of the invention.

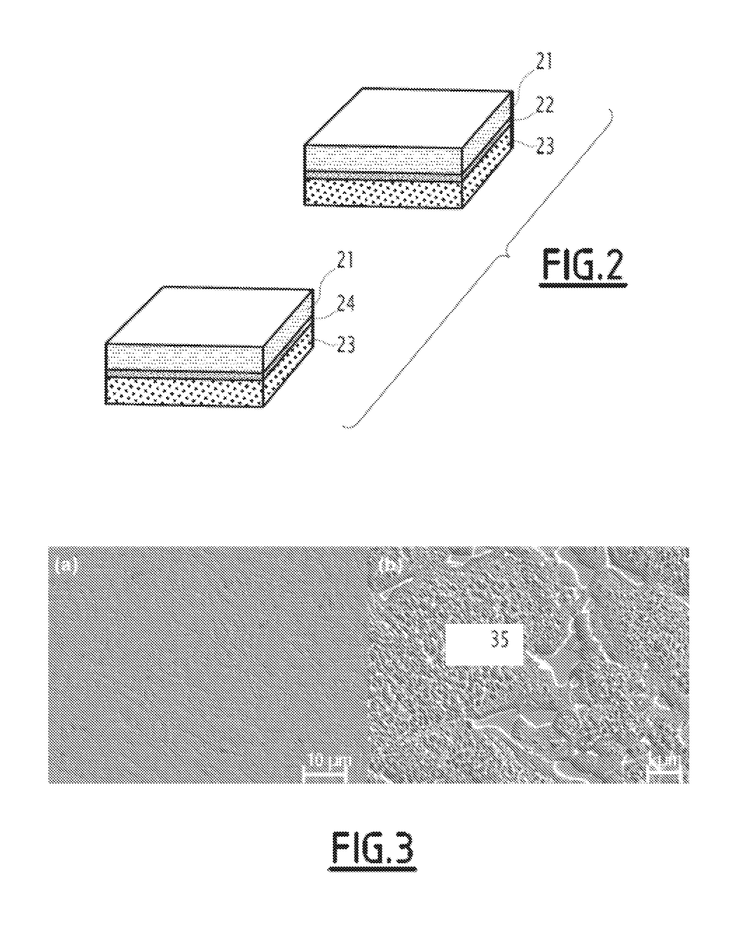

FIG. 2 diagrammatically shows two three-layer materials according to the invention comprising a layer of calcium phosphate (21), an intermediate layer of titanium nitride (22) or titanium oxide (24), and a layer of titanium or titanium alloy substrate (23).

### Example 1

### Preparation of Material According to the Invention by Chemical Treatment

For the examples, titanium, in particular the Ti6Al4V alloy, was used. Other metals or alloys may be used as substrate.

The preparation comprises four main steps, namely:
mechanical polishing, chemical etching using a modified Kroll's reagent (table 1);
the substrate is next pretreated with an alkaline solution (NaOH); then
undergoes a heat treatment; lastly
the pretreated material is submerged in an oxidizing, alkaline or acid autocatalytic bath, for 2 hours under defined temperature and pH conditions (table 2).

This principle is illustrated in FIG. 1(a).

Mechanical Polishing

A commercially available titanium alloy with a high titanium content (Ti6Al4V) in the form of a cylindrical bar for dental application was cut into small blocks (Ø 20 mm, height 2 mm). The titanium samples were polished by abrasion under a water jet using an automatic polishing device. The polishing disc of the device was placed under planetary rotation at 250 revolutions per minute with a polishing pressure of 10 N to 20 N. The titanium alloy slug is therefore moved at 250 rpm on the polishing disc. A series of polishing steps is carried out by refining the grit (grit 1000, 1200, 2500, 4000) for 2 minutes, until the surface state has the desired roughness. A suspension of amorphous colloidal silica for polishing (MasterMet 2, Buehler, Ill., USA) was used for final polishing of the titanium alloy samples. Lastly, the materials were cleaned separately by successive 15 minute ultrasonic treatments in acetone, then ethanol 70%, followed by two treatments with distilled water lasting 15 minutes each. The substrate had an arithmetic average roughness Ra (μm) of 0.16 and a maximum roughness Rmax (μm) of 0.73.

Chemical Etching

All of the samples were etched to remove the native oxides from the surface. The materials were placed in contact, for 2-5 minutes, with Kroll's reagent (mixture of 2 mL of hydrofluoric acid (HF, 40%), 4 mL of nitric acid (HNO₃, 66%) in 1000 mL of deionized water), then rinsed twice in distilled water. The surface state obtained after this step was observed by field emission scanning electron microscope (FESEM) and is shown in FIG. 3: (a) represents the surface state after treatment, (b) shows a micro-cross-linked structure background with vanadium islands (35), shown in FIG. 3(b).

TABLE 1
Composition of Kroll's reagent.

| Etchant | Composition | Concentration | Conditions |
|---|---|---|---|
| Kroll's Reagent | Distilled water | 1000 mL | 2 to 5 min |
| | HNO₃, 66% | 4 mL | |
| | HF, 40% | 2 mL | |

Alkaline and Heat Pretreatment

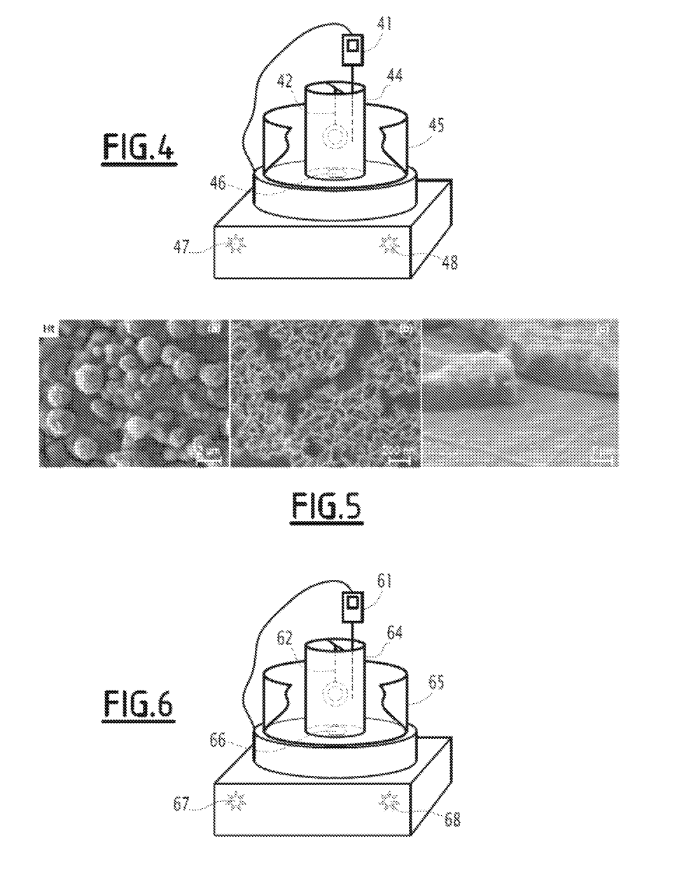

The titanium alloy materials are pretreated in an alkaline solution of 10 m NaOH at 60° C. for 24 hours in a Teflon® vial. FIG. 4 diagrammatically shows the equipment used for this treatment.

The samples are next washed with bidistilled water, then dried.

Next, the samples undergo a heat treatment at a temperature of 630° C. with a temperature ramp of 10° C./min, and maintained for 1 hour at 630° C. The materials are next left cool to ambient temperature (about 20° C.) in the furnace, then removed and kept in a drier for later analysis.

FIG. 5 shows the surface state of samples with spherical agglomerates of different sizes, but leaving a cellular nanometric structure visible (a). A highly nano-cross-linked structure is visible in FIG. 4(b). FIG. 4(c) shows a sample examined at a 50° angle to show the thickness of the cellular nanometric layer.

The coating is therefore made up of a heterogeneous surface of spherical agglomerates of 1-2 μm in approximate diameter (FIG. 5a) deposited on a nano-porous structure similar to that of the bone (pore diameter<100 nm) (FIG. 5b). The chemical and heat treatment allows the formation of a layer with a thickness of approximately 1.8 μm (FIG. 5c) containing Na⁺ and Ti⁴⁺ ions to form a layer of sodium titanate (Na₂Ti₅O₁₁).

This treatment allows hydroxyapatite nucleation and growth on the titanium pretreated with the sodium hydroxide solution.

Autocatalytic Depositions

To produce the layer of calcium phosphate, different baths have been used: one oxidizing, another acid, then another alkaline.

Each treatment was done for different lengths of time: 2 hours, 8 hours, 16 hours and 21 hours. The chemical composition is reported in table 2.

The calcium chloride makes it possible to provide the calcium and pyrophosphate and/or the sodium hypophosphite provides the phosphorus. Furthermore, sodium, pyrophosphate and sodium hypophosphite are reducing agents in an oxidizing or acid medium, respectively. In an acid medium, the succinic acid acts as a reaction accelerator, while the sodium fluoride is an etching agent. The catalyst used for the baths was either palladium chloride (PdCl₂) or silver chloride (AgCl).

FIG. 6 diagrammatically shows the device used for the autocatalytic deposition.

TABLE 2
Chemical composition for autocatalytic deposition.

| | | | | | Temper- |
|---|---|---|---|---|---|
| | | | Concen- | | ature of |
| | | | trations | | the bath |
| Bath | Reagents | | [g/L] | pH | [° C.] |
| Oxi- | Calcium | CaCl₂ | 5.6 | NaOH: | 60 ± 2 |
| dizing | chloride | | | 7.0 ± 0.1 | |
| | Sodium | NaP₂O₇•10H₂O | 6.7 | | |
| | pyro- | | | | |
| | phosphate | | | | |
| | Hydrogen | H₂O₂ | 34 | | |
| | peroxide | | | | |
| | Palladium | PdCl₂ or AgCl | 0.9 | | |
| | chloride | | | | |
| | or silver | | | | |
| | chloride | | | | |
| Acid | Calcium | CaCl₂ | 21.0 | NaOH: | 80 ± 2 |
| | chloride | | | 5.3 ± 0.1 | |
| | Sodium | NaF | 5.0 | | |
| | fluoride | | | | |
| | Succinic | C₄H₆O₄ | 7.0 | | |
| | acid | | | | |
| | Sodium | NaH₂PO₂•H₂O | 24.0 | | |
| | hypo- | | | | |
| | phosphite | | | | |
| | Palladium | PdCl₂ or AgCl | 0.885 | | |
| | chloride | | | | |
| | or silver | | | | |
| | chloride | | | | |
| Alka- | Calcium | CaCl₂ | 25.0 | NaOH: | 60 ± 2 |
| line | chloride | | | 9.2 ± 0.1 | |
| | Sodium | NaP₂O₇•10H₂O | 50 | | |
| | pyro- | | | | |
| | phosphate | | | | |
| | Sodium | NaH₂PO₂ H₂O | 21.0 | | |
| | hypo- | | | | |
| | phosphite | | | | |
| | Palladium | PdCl₂ or AgCl | 0.885 | | |
| | chloride | | | | |
| | or silver | | | | |
| | chloride | | | | |

The surface morphology of the samples was observed by FESEM after a carbon film was deposited on the surface.

The electron (Scanning Electron Microscopy)-material (surface to be analyzed) reaction leads to charge accumulation effects on the surface. These charges are discharged toward the ground in the case of a conductive sample. However, in the case of an insulator (such as the intermediate layer according to the invention), their accumulation deforms the electron beam and modifies its effective energy: it is therefore necessary to deposit a thin metallization layer on the surface (or carbon). Carbon has been chosen. This layer is therefore only deposited for SEM (FESEM) observation purposes.

FIG. 7 shows a deposit example, formed after 2 hours of treatment in an oxidizing (Ox), acid (Ac), or alkaline (Al) bath. The deposits in oxidizing and alkalizing baths have surfaces with structures similar to that observed by alkaline chemical and heat treatment (FIG. 5), indicating a potential to maintain proteins and antibiotics in the structure, beneficial to improve recovery or postsurgical healing. The surfaces obtained by alkaline bath have wide spherical agglomerates deposited on a layer of small spheroids formed on the metal substrate (diameter smaller than 50 nm), thereby suggesting a denser structure.

The chemical composition of the formed layers, analyzed by energy-dispersive spectroscopy (EDS-X), shows the presence of calcium and phosphorus. They are generated by the composition of the baths. Additionally, the fluoride detected with the use of the acid autocatalytic bath should improve the formation of bone at the interface when it is implanted on a bone site.

FIG. 7 shows the surfaces observed by FESEM after 2 hours of treatment in an oxidizing (a), acid (b), or alkaline (c) bath.

### Example 2

### Preparation of the Material According to the Invention by PLD

The principle of PLD physical deposition, then chemical deposition by autocatalytic deposition, comprises four main steps, which may be summarized as follows:
mechanical polishing (according to the polishing step of example 1)
chemical etching and ionic cleaning
PLD deposition
submersion of the materials in an autocatalytic bath (according to example 1).
   This principle is shown in FIG. 1(b).

Chemical Etching

The experimental chemical treatment consists of:
optional pretreatment of the samples by submersion in a sodium hydroxide (NaOH) and oxygen peroxide (H₂O₂) solution at 75° C. for 10 to 30 minutes to clean and decontaminate the surface of the titanium alloy of any coating particles and machining impurities.
treatment for 30 minutes in oxalic acid at 85° C. to produce a microporous surface;
optional final passivation in a nitric acid solution;
final cleaning is done using ions.

### PLD Deposition

A titanium dioxide layer of 300 nm (TiO₂) or a titanium nitride layer of 300 nm (TiN) was deposited on the titanium alloys by PLD to improve the adhesion and antimicrobial properties of the material.

To that end, the depositions were done by pulses generated by Quantel YAG laser (λ=355 nm). The laser source was placed outside the radiation chamber. The size of the radiation spot was about 2 mm² and the incident creep was 1.5 J/cm².

The titanium alloy sample was mounted on a special holder that could be rotated and/or translated during the application of the multi-pulse laser radiation to avoid piercing and continuously subject a new area to laser exposure. During the exposure, the titanium alloy substrate was kept at a temperature of about 600° C.

The external parameters are summarized in table 3.

TABLE 3
Experimental PLD conditions for deposition of TiN or TiO₂ films.

| | Substrate | Dynamic | DP during | | | | |
|---|---|---|---|---|---|---|---|
| Deposited | temperature | pressure | ablation | Energy | Focus | Laser | Deposition |
| substrate | [° C.] | (DP) [mbar] | [mbar] | [mJ] | [mm] | pulses | time [min] |
| TiO₂ | 602 | N₂ 1.2 * 10⁻² | MW + plasma | 10 | 690 | 60000 | 1 h 40 |
| TiN | 602 | O₂ 1.2 * 10⁻² | MW + plasma | 10 | 690 | 60000 | 1 h 40 |

MW (microwave) + plasma: heat treatment with microwave (MW) and “cleaning” with plasma to degas the surface of any organic residues.

Deposition by Autocatalytic Bath

A procedure identical to that of example 1 was done. In order to produce calcium phosphate layers, the samples were submerged in autocatalytic baths of different compositions summarized in table 2. FIG. 8 illustrates an intermediate layer of titanium nitride, and FIG. 9, of titanium dioxide, observed by FESEM.
Al: surface obtained by treatment with an alkaline bath (a);
Ac: surface obtained by treatment with an acid bath (b);
Ox: surface obtained with treatment with an oxidizing bath (c).

The submersion was done for 2 hours.

A heterogeneous structure of calcium and calcium phosphate of the intermediate layer was observed by EDS-X/FESEM (energy-dispersive analysis coupled with scanning electron microscopy) after treatment with an alkaline bath (FIG. 8a, 9a) and acid bath (FIG. 8a, 9a) on TiO₂ and TiN. Treatment with an oxidizing bath makes it possible to obtain a dense and uniform layer of calcium phosphate (FIG. 8c, 9c).

EDS-X analysis spectrums are obtained showing the presence of O, Na Ca, P for the acid and alkaline bath, and the presence of Cl and absence of Na for the oxidizing bath.

### Example 3

### Preparation of Material According to the Invention by Developing the Calcium Phosphate Layer by Sol Gel

The principle of deposition using the sol gel method comprises four main steps, which can be summarized as follows:
mechanical polishing,
chemical etching,
preparation of a calcium phosphate gel; and
deposition of the gel on the etched substrate.

The substrate is prepared according to steps (i), (ii) and (iii) of example 1.

A sol gel suspension of calcium phosphate is prepared under the following conditions (according to C. Wen, W. Xu, W. Hu, and P. Hodgson, “Hydroxyapatite/titania sol-gel coatings on titanium-zirconium alloy for biomedical applications,” Acta Biomaterialia, vol. 3, no. 3, pp. 403-410, May 2007):

The following components are mixed at temperatures comprised between 20° C. and 100° C.:
Calcium nitrate tetrahydrate (Ca(NO₃)₂.4H₂O)
Triethyl phosphite (P(C₂H_{S}O)₃)
Ethanol
Distilled water

The Ca/P molar ratio is equal to 1.67.

A triethyl phosphite solution with a concentration of 1.8M is prepared in anhydrous ethanol. A quantity of distilled water corresponding to a water/phosphite molar ratio comprised between 1 and 6, preferably between 3 and 4, is added. The whole is subjected to agitation for 24 hours in a beaker, preferably made from Teflon, and closed.

A solution of calcium nitrate tetrahydrate in anhydrous ethanol with a concentration comprised between 2 and 4 M is added, drop by drop, to the preceding solution.

The mixture is agitated for 3 minutes to 1 hour and aged at ambient temperature for up to 3 days.

### Example 3.1

### Deposition by Spin Coating

The preceding mixture is deposited by spin coating at a speed of 3000 revolutions per minute for 15 seconds to 2 minutes, preferably 15 to 40 seconds. The substrate is next treated between 400° C. and 700° C. from 5 minutes to 1 hour, preferably between 500° C. and 630° C. for 20 minutes, in an argon/air atmosphere. The obtained layer of calcium phosphate has a thickness of about 1 μm. The method can be repeated several times to obtain a thicker layer of calcium phosphate.

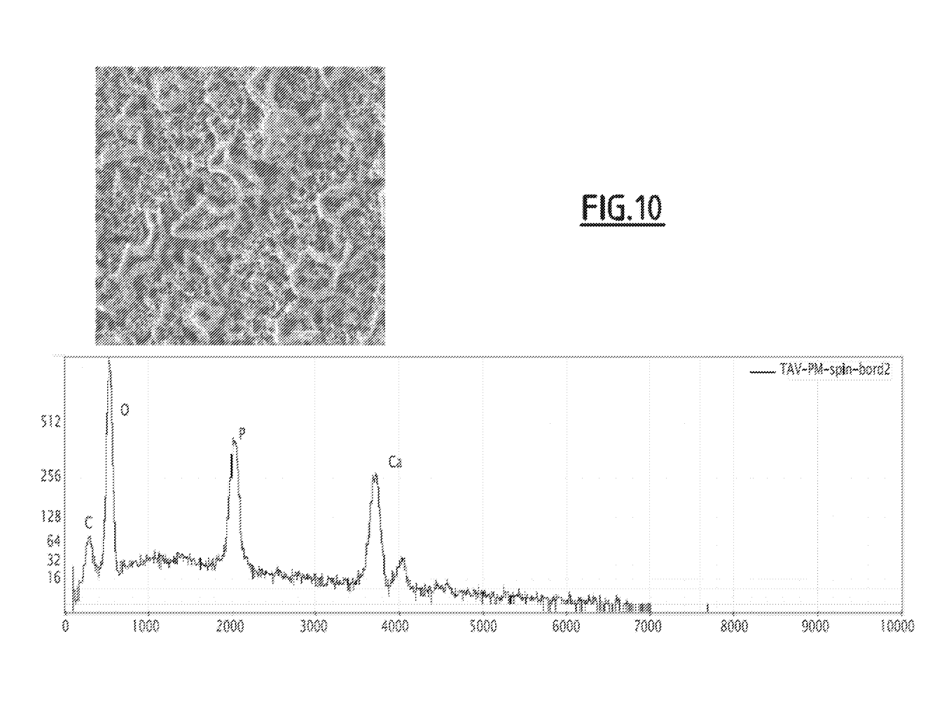

The substrates are next cleaned by ultrasound in acetone, next in ethanol, then in distilled water. The dense layer of calcium phosphate can be seen in FIG. 10 by FESEM, as well as the EDS-X composition analysis.

### Example 3.2

### Deposition by Dip Coating

The substrate is dipped in the preceding mixture at a speed comprised between 1 and 20 cm/minute (preferably 3-10 cm/minute), then treated between 400° C. and 700° C. from 5 minutes to 1 hour, preferably between 500° C. and 630° C. for 20 minutes, in an argon/air atmosphere. The thickness of the obtained calcium phosphate layer is several micrometers. The method may be repeated several times to obtain a thicker layer of calcium phosphate.

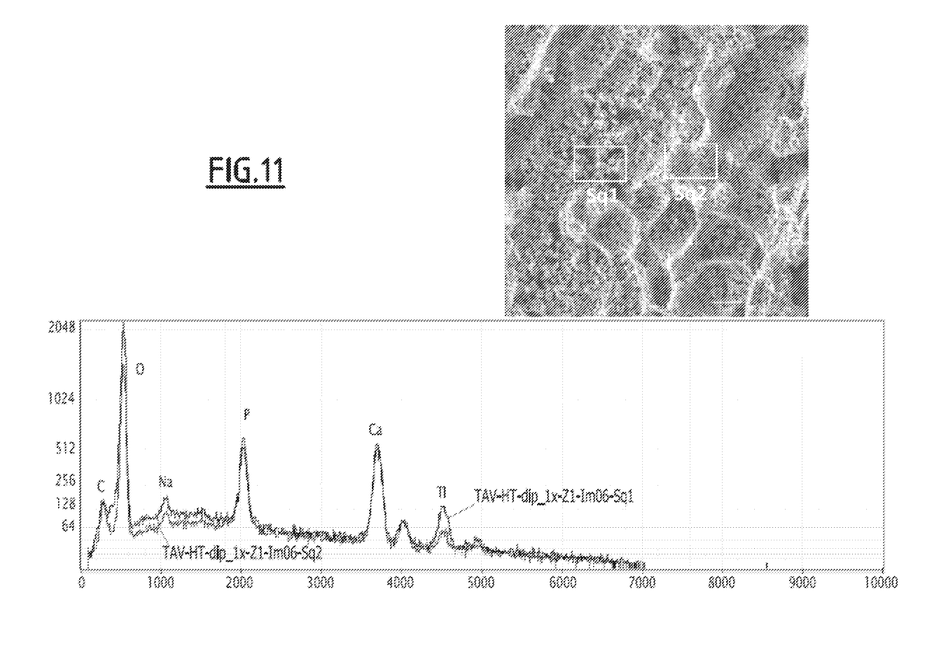

The substrates are next cleaned by ultrasound in acetone, next in ethanol, then in distilled water. The dense layer of calcium phosphate can be seen in FIG. 11 by FESEM as well as the EDS-X composition analysis.

### Example 4

### Osteoblast Viability Study

A viability study was done for the osteoblasts on the samples developed as in table 4 below:

The control (100%) corresponds to the activity of the mitochondrial dehydrogenase of the cultivated cells on a traditional plastic used for cell growth and the surface area of which is ideal for cell growth.

Cell Culture

Human osteosarcoma cells (Human osteosarcoma cells; MG63, ATCC: CRL-1427) were cultivated at 37° C., in a modification minimal essential medium (5% CO₂ in Dulbecco's modification minimal essential medium; DMEM, Sigma-Aldrich, St. Louis, Mo., USA) in the presence of fetal bovine serum (10% fetal bovine serum; Lonza, Basel, Switzerland) and 1% antibiotics (penicillin-streptomycin). When the cells reached 85-90% confluence, they were detached by trypsin (Sigma-Aldrich, St. Louis, Mo.), collected [and] used for cytotoxicity evaluations. The samples with a layer of calcium phosphate were sterilized by submersion in 70% ethanol for 12 hours and were next dried in a sterile chamber and radiated by UV light exposure for 45 minutes.

Cytotoxicity Evaluation

The samples were deposited in the wells of 24-well plates (CellStar, PBI International, Milan, Italy). The cells were inoculated directly onto the surface of the samples in a defined number (5000 cells/sample) and cultivated for 48 hours and 72 hours. The cells inoculated on polystyrene were used as a control.

Cell viability was evaluated by treatment with MTT (3-(4.5-Dimethyl-2-thiazolyl)-2.5-diphenyl-2H-tetrazolium bromide assay (MTT, Sigma-Aldrich St. Louis, Mo., USA). Briefly, 20 mL of a MTT solution (1 mg/ml in PBS) was added to each sample and each plate, and incubated for 4 hours in a dark place. Afterwards, the supernatant was suctioned and the formazan crystals were dissolved with 100 mL of dimethyl sulfoxide (DMSO, Sigma-Aldrich). 50 mL was collected, centrifuged for 5 minutes (12,000 rpm) to eliminate any debris. The optical density was measured at a wavelength of 570 nm with a spectrophotometer (Spectra Count, Packard Bell, USA). The optical density of the control samples corresponds to a value of 100% cell viability.

TABLE 4

| Treatment | Substrate | | | | | | | |
|---|---|---|---|---|---|---|---|---|
| Substrate material | TAV-laboratory | | | TAV-commercial | | | 316L | polymer |
| | | | | | | | | PP-PE |
| | | | | | | | | polypropylene- |
| | | | | | | | | polyethylene |
| Polishing + Kroll | Yes | | | yes | | | yes | no |
| chemical etching | | | | | | | | |
| (example 1) | | | | | | | | |
| Alkaline | Yes | | | yes | | | yes | yes |
| pretreatment - | | | | | | | | |
| NaOH (example 1) | | | | | | | | |
| Heat treatment | 630° C., 1 h | | | 630° C., 1 h | | | no | no |
| (example 1) | | | | | | | | |
| Autocatalytic | acid | alkaline | oxidizing | acid | alkaline | oxidizing | acid | acid |
| deposition | | | | | | | | |
| (example 1) | | | | | | | | |
| (catalyst) | AgCl | | | PdCl₂ | | | PdCl₂ | PdCl₂ |
| Length of the bath | 2 h | 2 h | 2 h | 3 h | 3 h | 3 h | 3 h | 3 h |
| Length of | 48 h | 48 h | 48 h | 48 h | 48 h | 48 h | 48 h | 48 h and 72 h |
| osteoblast growth | | | | | | | and | |
| study | | | | | | | 72 h | |

TAV: alloy of Ti6—Al—V4.

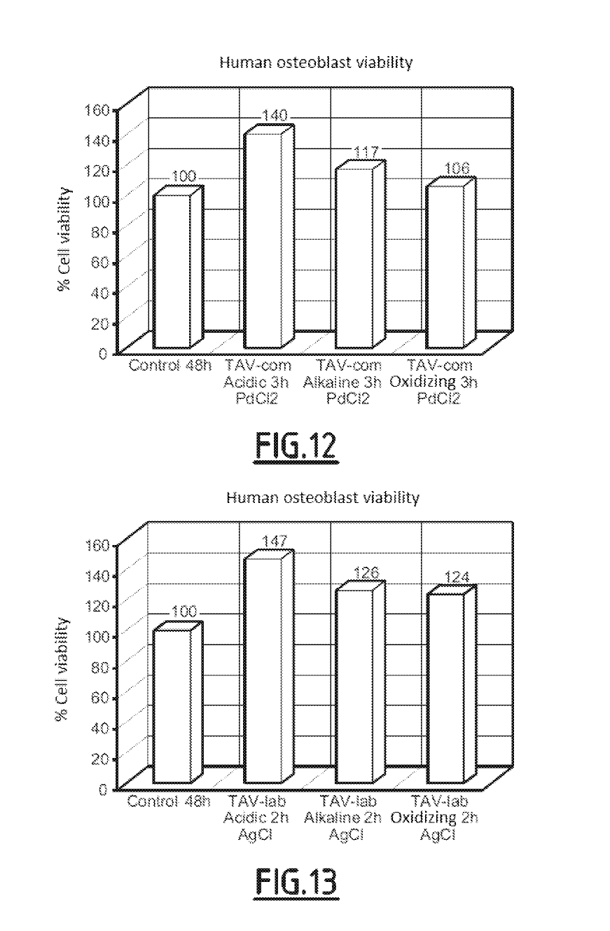

FIGS. 12 and 13 show the cell viability on TAV (commercial) substrates treated by autocatalytic baths lasting three hours with PdCl₂ as catalyst (FIG. 12) and lasting 2 hours with AgCl as catalyst (FIG. 13).

Values above 100% mean that the cells feel better on the “implants” than on plastic.

Very good osteoblast growth is observed on the surface of the composite materials of the invention. Better growth is noted during the use of an acid autocatalytic bath independently of the catalyst used. It will also be noted that the catalyst of the AgCl type makes it possible to obtain better growth results.

1. A multi-layer material comprising a metal or metal alloy substrate, the metal or alloy substrate being coated with an intermediate layer comprising at least one ceramic or crystalline, or partially crystalline, structure, including a metal or a metal alloy, said intermediate layer being coated with a layer of calcium phosphate having a cellular nanometric structure.

2. The material according to claim 1, wherein said metal or metal alloy substrate has a roughness of less than 800 nm.

3. The material according to claim 1, wherein the intermediate layer comprises or is made up of sodium titanate (Na2Ti5O11), titanium dioxide, and/or titanium nitride.

4. The material according to claim 3, wherein the intermediate layer has a thickness of 50 nanometers to 10 micrometers.

5. The material according to claim 3, wherein the intermediate layer has a thickness of 100 to 500 nm.

6. A method for preparing a multilayer material comprising a metal or metal alloy substrate, and an intermediate layer comprising a ceramic or crystalline, or partially crystalline, structure including a metal or a metal alloy, wherein said method comprises:
(i) mechanical polishing of a metal or metal alloy substrate,
(ii) chemical etching to remove any native surface oxides of the substrate;
(iii) producing an intermediate layer comprising at least one ceramic or crystalline, or partially crystalline, structure, including a metal or metal alloy on the surface of the substrate; and
(iv) depositing on the intermediate layer of the material obtained in step (iii), a layer of calcium phosphate comprising a cellular nanometric surface structure.

7. The method according to claim 6, comprising in steps (ii) and (iii):
(a1) chemical etching to remove the native surface oxides by putting the polished surface in contact with an aqueous solution of hydrochloric acid and nitric acid;
(b1) placing the material in contact, for example by submersion, with an alkaline solution to generate a deposit, on the surface of the substrate, of an intermediate layer of a ceramic or crystalline, or partially crystalline, structure, including a metal or a metal alloy, and preferably titanium, then preferably washing and drying the material; and
(c1) heating the material.

8. The method according to claim 6, comprising in steps (ii) and (iii):
(a2) chemical etching to remove the native surface oxides, refine the porosity and passivate the surface of the substrate, to prepare the surface of the substrate for step (b2); and
(b2) producing a layer of a ceramic or crystalline, or partially crystalline, structure, including a metal or a metal alloy, and preferably titanium, by pulsed laser deposition (PLD) on the surface of the substrate.

9. The method according to claim 6, wherein step (iv) is done by placing the material in a solution comprising calcium and phosphate ions for autocatalytic deposition on the intermediate layer of a calcium phosphate layer comprising a cellular nanometric structure on the surface; or this is done by depositing a calcium phosphate sol gel on the intermediate layer to obtain a calcium phosphate layer comprising a cellular nanometric structure on the surface.

10. The method according to claim 6, comprising growing the calcium phosphate layer by placing the material in contact with a simulated body fluid (SBF).

11. The method according to claim 6, wherein the autocatalytic bath comprises an oxidizing bath, an acid bath or an alkaline bath.

12. The method according to claim 6, wherein step (iv) is done: (a) at a temperature between 50° C. and 70° C., and in an alkaline bath; or (b) at a temperature between 60° C. and 80° C., in an oxidizing bath; or (c) at a temperature between 70° C. and 90° C., in an acid bath.

13. The method according to claim 8, wherein step (b2) comprises producing a layer of 100 to 500 nm of metal nitride or dioxide by pulsed laser deposition (PLD) on the surface of the substrate.

14. A multi-layer material that may be obtained according to the method described in claim 6.

15. An implant or prosthesis for a bone or dental structure comprising a material as defined in claim 1.

16. (canceled)

17. A method of treating a human being comprising surgically implanting an implant according to claim 15 in said human.

18. The method according to claim 17, wherein the implant is used to replace an articular bone end or for dental surgery.

19. The material of claim 1, wherein said metal or metal alloy substrate has a roughness of less than 500 nm.

20. The method of claim 9, wherein said placing the material in a solution comprising calcium and phosphate ions comprises submersion of the material in the solution.

21. The method of claim 12, wherein said alkaline bath has a pH of between 8 and 10.

22. The method of claim 12, wherein said oxidizing bath has a pH of about 7.

23. The method of claim 12, wherein said acid bath has a pH of between 4 and 6.

24. The method of claim 13, wherein said metal nitride or dioxide is titanium nitride or titanium dioxide.

25. The method of claim 18, wherein said articular bone end is in a hip, knee, shoulder, elbow, ankle, wrist, finger or toe.
